SEQUENCE LISTING (4 sequences)

SEQ ID NO: 1 - DNA, 22 bp, synthetic construct
  source 1..22: mol_type = other DNA; note = forward primer for detecting TGF-beta1
tgatacgcctgagtggctgtct

SEQ ID NO: 2 - DNA, 22 bp, synthetic construct
  source 1..22: mol_type = other DNA; note = reverse primer for detecting TGF-beta1
cacaagagcagtgagcgctgaa

SEQ ID NO: 3 - DNA, 23 bp, synthetic construct
  source 1..23: mol_type = other DNA; note = forward primer for detecting GAPDH
catcactgccacccagaagactg

SEQ ID NO: 4 - DNA, 23 bp, synthetic construct
  source 1..23: mol_type = other DNA; note = reverse primer for detecting GAPDH
atgccagtgagcttcccgttcag

(19) United States
(12) Patent Application Publication
(10) Pub. No.: US 2026/0083825 A1
(43) Pub. Date: Mar. 26, 2026

(54) COMPOSITION FOR PROMOTING WOUND HEALING AND METHOD FOR PROMOTING WOUND HEALING USING THE SAME

(71) Applicant: TAICEND TECHNOLOGY CO., LTD., Kaohsiung City (TW)

(72) Inventors: Yu-Pu Chu, Kaohsiung City (TW); Yi-Meng Chang, Pingtung City (TW)

(21) Appl. No.: 18/892,124

(22) Filed: Sep. 20, 2024

(51) Int. Cl. A61K 38/39; A61K 31/198; A61P 17/02

(52) U.S. Cl. CPC A61K 38/39; A61K 31/198; A61P 17/02

(57) ABSTRACT

A composition for promoting wound healing includes an amino acid mixture and a fish collagen present in a weight ratio ranging from 1:3 to 3:1. The amino acid mixture includes glycine and proline. A method for promoting wound healing which includes administering to a subject in need thereof the composition is also provided.

### SEQUENCE LISTING XML

The Sequence Listing submitted concurrently herewith with a file name of “PE-70231-AM-SEQUENCE LISTING.xml,” a creation date of Jun. 21, 2024, and a size of 5.07 kilobytes, is part of the specification and is incorporated by reference in its entirety.

### FIELD

The disclosure relates to a composition for promoting wound healing, and a method for promoting wound healing using the same.

### BACKGROUND

During the process of wound healing, fibroblasts may gather at and attach to a wound site for proliferation, and then secrete growth factors, cytokines, collagens and extracellular matrix components, which in turn leads to angiogenesis, epithelialization, and collagen remodeling, thereby promoting wound healing.

It has been reported that fish collagen may be applied in wound dressing. For instance, Jafari H. et al. (2020), Polymers (Basel), doi: 10.3390/polym12102230 discloses that collagen-based wound dressing from the scales of tilapia and grey mullet showed excellent antimicrobial activity, and such wound dressing exhibited high wound closure capacity, indicating that fish scale collagen is capable of accelerating re-epithelialization.

In spite of the aforesaid, there is still a need to develop a product with excellent effectiveness in promoting wound healing.

### SUMMARY

Accordingly, in a first aspect, the present disclosure provides a composition for promoting wound healing, which can alleviate at least one of the drawbacks of the prior art. The composition includes an amino acid mixture and a fish collagen present in a weight ratio ranging from 1:3 to 3:1. The amino acid mixture includes glycine and proline.

In a second aspect, the present disclosure provides a method for promoting wound healing, which can alleviate at least one of the drawbacks of the prior art. The method includes administering to a subject in need thereof the aforesaid composition.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the cell attachment percentage determined in each group as described in Section A of Example 2, infra, in which the symbols “*”, “**”, “***”, and “****” represent p<0.05, p<0.01, p<0.001, and p<0.0001, respectively (compared with the control group).

FIG. 2 shows the expression level of TGF-β1 gene determined in each group as described in Section A of Example 2, infra, in which the symbols “***” and “****” represent p<0.001 and p<0.0001, respectively (compared with the control group).

FIG. 3 shows the wound healing percentage determined in each group as described in Section C of Example 2, infra, in which the symbols “*” and “****” represent p<0.05 and p<0.0001, respectively (compared with the control group).

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

By conducting research, the applicant found that when an amino acid mixture, which includes glycine and proline, and a fish collagen are used in combination in a weight ratio ranging from 1:3 to 3:1, such combination of the amino acid mixture and the fish collagen is effective in promoting attachment of fibroblasts and secretion of transforming growth factor-β1 (TGF-β1) by fibroblasts, and also exhibits excellent effect in promoting fibroblast-mediated wound closure, and hence can be expected to be applied in wound healing.

Therefore, the present disclosure provides a composition for promoting wound healing, which includes an amino acid mixture and a fish collagen present in a weight ratio ranging from 1:3 to 3:1. The amino acid mixture includes glycine and proline.

In certain embodiments, the amino acid mixture and the fish collagen are present in a weight ratio of 1:1.

According to the present disclosure, the fish collagen may be a commercially available product, or may be extracted from a fish skin using an extraction method well known to those skilled in the art. In this regard, those skilled in the art may refer to journal articles, e.g., Jafari H. et al. (2020), supra.

According to the present disclosure, the fish species suitable for use as a source of fish skin is not particularly limited. In certain embodiments, the fish species may be selected from the group consisting of Oreochromis mossambicus (also knowns as tilapia), Oreochromis niloticus (also known as Nile tilapia), Oreochromis aureus (also knowns as Tilapia aurea), Oreochromis niloticus×Oreochromis aureus, and combinations thereof. In an exemplary embodiment, the fish collagen is extracted from the fish skin of Oreochromis niloticus×Oreochromis aureus.

According to the present disclosure, the glycine and the proline are present in a weight ratio ranging from 1:3 to 3:1. In an exemplary embodiment, the glycine and the proline are present in a weight ratio of 2:1.

As used herein, the term “wound” is intended to cover various wound types, which may include, but are not limited to: a chronic wound, such as a decubitus ulcer (also known as pressure ulcer), a diabetic ulcer (e.g., a diabetic foot ulcer), a venous ulcer (e.g., a venous leg ulcer), an arterial ulcer, an infectious ulcer, a gastrointestinal ulcer, a surgical wound, and a burn; and an acute wound (e.g., a cut, a scrape, and a tear).

As used herein, the terms “wound healing” and “wound repair” can be interchangeably used.

The present disclosure also provides a method for promoting wound healing, which includes administering to a subject in need thereof the aforesaid composition.

As used herein, the terms “administering” and “administration” can be interchangeably used, and mean introducing, providing or delivering a pre-determined active ingredient (e.g., the above-mentioned pharmaceutical composition) to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

In certain embodiments, the composition may be formulated as a pharmaceutical composition.

According to the present disclosure, the pharmaceutical composition may be formulated into a dosage form suitable for topical administration using technology well known to those skilled in the art.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, adsorption delaying agent, liposomes, and the like. The choice and amount of the aforesaid agents are within the expertise and routine skills of those skilled in the art.

According to the present disclosure, the pharmaceutical composition may be formulated into an eternal preparation suitable for topical application to the skin using technology well known to those skilled in the art. The external preparation includes, but is not limited to, emulsions, gels, ointments, creams, patches, liniments, powder, aerosols, sprays, lotions, serums, pastes, foams, drops, suspensions, salves, dressings, and bandages.

According to the present disclosure, the external preparation may be produced by mixing the above-mentioned pharmaceutical composition with a base well known to those skilled in the art.

According to the disclosure, the base may include one or more of the followings additives: water, alcohols, glycols, hydrocarbons (such as petroleum jelly and white petrolatum), waxes (such as paraffin and yellow wax), preserving agents, antioxidants, surfactants, absorption enhancers, stabilizing agents, gelling agents (such as Carbopol® 941, microcrystalline cellulose, carboxymethyl cellulose), active agents, humectants, odor absorbers, fragrances, pH adjusting agents, chelating agents, emulsifiers, occlusive agents, emollients, thickeners, solubilizing agents, penetration enhancers, anti-irritants, colorants, propellants, and so forth. The choice and amount of the aforesaid additives are within the expertise and routine skills of those skilled in the art.

According to the present disclosure, the dose and frequency of administration of the pharmaceutical composition may vary depending on the following factors: the type, location, area, depth and severity of a wound to be treated, the level of wound exudate, the degree of wound healing, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### Examples

### General Experimental Materials:

1. The glycine powder and the proline powder used in the following examples were purchased from Merck & Co., Inc.
2. Source and cultivation of mouse embryonic fibroblast cell line NIH/3T3:

The mouse embryonic fibroblast cell line NIH/3T3 (BCRC 60008) used in the following examples was purchased from the Bioresource Collection and Research Center (BCRC) of the Food Industry Research and Development Institute (FIRDI) (No. 331, Shih-Pin Rd., Hsinchu City 300, Taiwan).

The NIH/3T3 cells were grown in a Petri dish containing a RPMI 1640 medium (Gibco) supplemented with 10% of fetal bovine serum (FBS) and 1% of penicillin-streptomycin (Gibco), followed by cultivation in an incubator with culture conditions set at 37° C. and 5% CO₂. Medium change was performed every two to three days. Cell passage was performed when the cultured cells reached 90% of confluence.

### General Procedures:

### 1. Statistical Analysis:

The experimental data obtained in all the test groups are expressed as mean±standard deviation (SD), and were analyzed using one-way analysis of variance (one-way ANOVA) followed by Tukey's post-hoc test and unpaired Student's t-test, so as to evaluate the differences of the groups. Statistical significance is indicated by p<0.05.

### Example 1. Preparation of Composition According to the Disclosure

### Experimental Materials:

### 1. Preparation of Amino Acid Mixture:

The glycine powder and the proline powder were mixed in a weight ratio of 2:1, so as to obtain an amino acid mixture containing glycine and proline.

### 2. Preparation of Fish Collagen:

The fish collagen was prepared generally according to the method described in Jafari H. et al. (2020), supra. Briefly, skins of Oreochromis niloticus×Oreochromis aureus obtained from Fortune Life Enterprise Co., Ltd. were pretreated by immersing in a sodium hydroxide solution, which was accompanied with stirring, for 24-36 hours, so as to remove lipids of the skins, followed by a rinse with a deionized water to remove remainder of the sodium hydroxide solution. Next, the treated fish skins were added with acetic acid, and were then stirred for 24 hours, so as to decompose cross-linked collagens. After filtration or centrifugation, an enzymatic hydrolysis reaction was conducted using pepsin for 12 hours to remove telopeptides, thereby obtaining a hydrolysate. Subsequently, the hydrolysate was subjected to salting-out, followed by centrifugation at 4° C. and 10000 rpm for 3 minutes, thereby obtaining a precipitate. After that, the precipitate was dissolved using acetic acid, followed by performing dialysis and drying in sequence, thereby obtaining the fish collagen in powder form.

### Experimental Procedures:

Each of compositions 1 to 3 was prepared by mixing the amino acid mixture and the fish collagen respectively described in Sections 1 and 2 of the Experimental materials of this example, and the corresponding recipe are shown in Table 1 below.

TABLE 1

| | |
|---|---|
| Amino acid mixture | Fish collagen |

| | |
|---|---|
| Composition | Amount (wt %) |

| | | |
|---|---|---|
| 1 | 75 | 25 |
| 2 | 50 | 50 |
| 3 | 25 | 75 |

### Example 2. Evaluation of the Efficacy of Composition According to the Disclosure in Promoting Wound Healing

### Experimental Procedures:

### A. Cell Attachment Assay:

First, the NIH/3T3 cells prepared in Section 2 of the General Experimental Materials were divided into 6 groups, including a control group, two comparative groups (i.e., comparative groups 1 and 2), and three experimental groups (i.e., experimental groups 1 to 3). Each group of the NIH/3T3 cells was seeded at a concentration of 6×10⁵ cells/well in a respective well of a 6-well culture plate containing 1000 μL of RPMI 1640 medium supplemented with 10% of FBS and 1% of penicillin-streptomycin. Next, each of the comparative groups 1 and 2, and experimental groups 1 to 3 was treated with a suitable amount of the corresponding treating agent according to Table 2 below, such that a final concentration of the treating agent in each group was 2 mg/ml. In addition, the control group received no treatment.

TABLE 2

| Group | Treating agent |
|---|---|
| Control group | — |
| Comparative group 1 | Amino acid mixture |
| Comparative group 2 | Fish collagen |
| Experimental group 1 | Composition 1 |
| Experimental group 2 | Composition 2 |
| Experimental group 3 | Composition 3 |

Next, each group of the NIH/3T3 cells was incubated in an incubator with culture conditions set at 37° C., 5% CO₂ for 6 hours. After that, the culture medium in each well was removed, and then the cell culture was washed twice with phosphate buffer saline (PBS), followed by adding 100 μL of trypsin and left to stand for 3 minutes, so as to detach the NIH/3T3 cells from a bottom of the 6-well culture plate. Afterwards, 900 μL of RPMI 1640 medium supplemented with 10% of FBS and 1% of penicillin-streptomycin was added to each well to neutralize the activity of the trypsin, and a resulting cell suspension thus formed was collected using a pipette, and then placed into a microcentrifuge tube, followed by conducting cell counting using techniques well-known to those skilled in the art. The cell attachment percentage (%) was calculated by substituting a number of attached cells, i.e., the NIH/3T3 cells obtained after the cell counting, into the following Equation (1):

$$A = ( B / C ) \times 100 \qquad ( 1 )$$

$$where A = cell attachment percentage ( \% )$$

$$B = number of the attached cells of each group$$

$$C = number of attached cells of the control group$$

The data thus obtained were analyzed according to the procedures as described in Section 1 of the General Procedures.

### B. Determination of Expression Level of TGF-β1 Gene:

After completing the cell attachment assay in Section A of this example, each group of the NIH/3T3 cells was subjected to total RNA extraction using TRIzol™ reagent (Invitrogen) in accordance with the manufacturer's instructions, so as to obtain total RNA. The total RNA of each group was used as a template for synthesizing cDNA by reverse transcription polymerase chain reaction (RT-PCR) using iScript™ Reverse Transcription Supermix (Bio-Rad, Cat. No. 1708840) in accordance with the manufacturer's instructions.

The thus obtained cDNA, serving as a DNA template, was subjected to quantitative real-time polymerase chain reaction (PCR) based on SYBR Green fluorescence, which was performed on a SensiFAST™ SYBR® No-ROX kit (Bioline, Cat. No. BIO-98020) using a designed primer pair specific for TGF-β1 gene shown in Table 3 and the reaction conditions shown in Table 4. Glyceraldehyde-3-phosphate dehydrogenase (GAPDH) gene was used as an endogenous control in the quantitative analysis of real-time PCR to normalize the gene expression data.

TABLE 3

| | | | Size of |
|---|---|---|---|
| | | | PCR |
| | | Nucleotide sequence | product |
| Target gene | PCR primer pair | (5′→3′) | (bp) |
| TGF-β1 gene | Forward primer | tgatacgcctgagtggctgtct | 107 |
| (NCBI Accession | (TGF-β1-F) | (SEQ ID NO: 1) | |
| Number | Reverse primer | cacaagagcagtgagcgctgaa | |
| NM_011577) | (TGF-β1-R) | (SEQ ID NO: 2) | |
| GAPDH gene | Forward primer | catcactgccacccagaagactg | 153 |
| (NCBI Accession | (GAPDH-F) | (SEQ ID NO: 3) | |
| Number | Reverse primer | atgccagtgagcttcccgttcag | |
| NM_008084) | (GAPDH-R) | (SEQ ID NO: 4) | |

TABLE 4

| Reaction mix | Volume (μL) |
|---|---|
| cDNA (20 ng/μL) | 1 |
| Forward primer (10 μM) | 0.5 |
| Reverse primer (10 μM) | 0.5 |
| Fast SYBR ® Green Master Mix | 5 |
| (Applied Biosystems) | |
| Deionized water | 3 |

Operation conditions: 39 cycles of the following reaction: denaturation at 95° C. for 5 seconds, annealing at 60° C. for 32 seconds, and extension at 65° C. for 5 seconds.

The resultant PCR product was subjected to determination of fluorescence intensity, followed by calculating the cycle threshold (Ct) value of TGF-β1 gene. The Ct value of TGF-β1 gene in each group was normalized with that of GAPDH gene using the comparative Ct method, and the relative fold change in TGF-β1 gene expression for each group was further calculated using the following Equation (2):

$$D = ( E - F ) / ( G - F ) \qquad ( 2 )$$

$$where D = relative fold change in TGF - β 1 gene expression$$

$$E = normalized {C}_{t} value of TGF - β 1 gene in each group$$

$$F = normalized {C}_{t} value of TGF - β 1 gene in the blank control group$$

$$G = normalized {C}_{t} value of TGF - β 1 gene in the Ca control group$$

The data thus obtained were analyzed according to the procedures as described in Section 1 of the General Procedures.

### C. Wound-Healing Assay:

First, the NIH/3T3 cells prepared as described in Section 2 of the General Experimental Materials were divided into 4 groups, including a control group and three experimental groups (i.e., experimental groups 1 to 3). Each group of the NIH/3T3 cells was seeded at a concentration of 6×10⁵ cells/well in a respective well of a 6-well culture plate containing 1000 μL of RPMI 1640 medium supplemented with 10% of FBS and 1% of penicillin-streptomycin. Next, each group of the NIH/3T3 cells was incubated in an incubator with culture conditions set at 37° C., 5% CO₂ for 24 hours.

After that, the cell culture of each group was scraped along the diameter of the corresponding well by using a pipette tip to create a cell-free wound area of approximately 600 μm. Afterwards, the culture medium in each well was removed, and the respective well was washed with PBS. After adding a fresh RPMI 1640 medium supplemented with 10% of FBS and 1% of penicillin-streptomycin, each of the cell cultures of the experimental groups 1 to 3 was treated with a suitable amount of the corresponding treating agent according to Table 5 below, such that a final concentration of the treating agent in each group was 2 mg/ml. In addition, the control group received no treatment.

TABLE 5

| Group | Treating agent |
|---|---|
| Control group | — |
| Experimental group 1 | Composition 1 |
| Experimental group 2 | Composition 2 |
| Experimental group 3 | Composition 3 |

After that, the cell culture of each group was incubated in an incubator with culture conditions set at 37° C., 5% CO₂ for 24 hours. Prior to incubation and on the 24^{th} hour after start of the incubation, the wound area in each group was subjected to observation using an inverted microscope (OLYMPUS; Model: CKX53) under a magnification of 100×, and then to photography using a digital camera (OLYMPUS; Model: OM-D). Each of the digital images thus obtained was subjected to analysis using Image J software, so as to calculate the size of wound area. The wound healing percentage (%) was calculated by substituting the thus obtained size of wound area into the following Equation (3):

$$H = [ 1 - ( I / J ) ] \times 100 \qquad ( 3 )$$

$$where H = wound healing percentage ( \% )$$

$$I = size of the would area on the {24}^{th} hour after start of incubation$$

The data thus obtained were analyzed according to the procedures as described in Section 1 of the General Procedures.

### Results:

### A. Cell Attachment Assay:

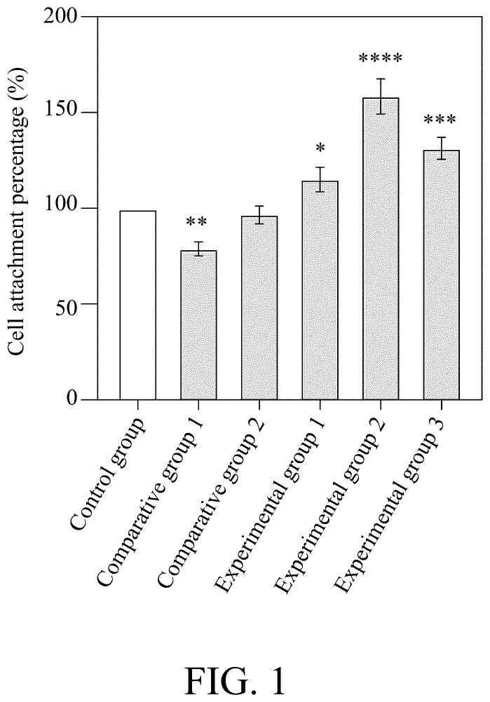

Referring to FIG. 1, the cell attachment percentage determined in each of the experimental groups 1 to 3 (treated with the compositions 1 to 3, respectively) was significantly increased compared with that determined in the control group, in which the cell attachment percentage determined in the experimental group 2 had the highest degree of increase. In addition, in comparison with the control group, the cell attachment percentage determined in the comparative group 2 (treated with the fish collagen) showed no significant difference, while that of the comparative group 1 (treated with the amino acid mixture) was significantly reduced. These results indicate that the composition according to the disclosure can exhibit satisfactory efficacy in promoting attachment of the NIH/3T3 cells. As for the amino acid mixture and the fish collagen, not only are they unable to promote attachment of the NIH/3T3 cells, but they may even cause an adverse effect.

### B. Determination of Expression Level of TGF-β1 Gene:

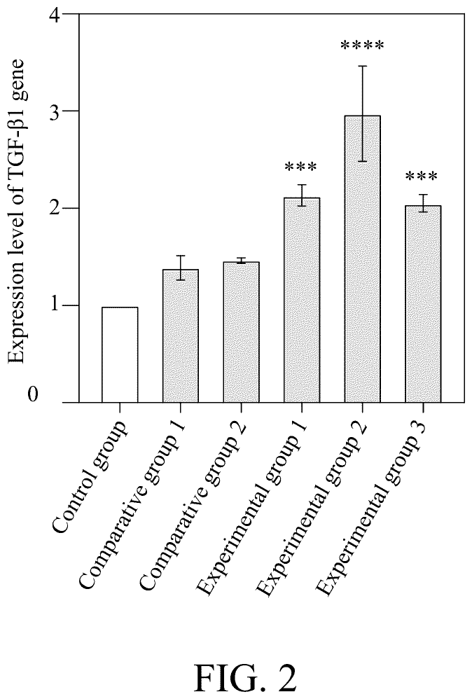

Referring to FIG. 2, the expression level of TGF-β1 gene determined in each of the experimental groups 1 to 3 (treated with the compositions 1 to 3, respectively) was significantly increased compared with that in determined in the control group, in which the expression level of TGF-β1 gene determined in the experimental group 2 had the highest degree of increase. In addition, in comparison with the control group, the expression level of TGF-31 gene determined in the comparative group 1 (treated with the amino acid mixture) or the comparative group 2 (treated with the fish collagen) showed no significant difference. These results indicate that the composition according to the disclosure can exhibit satisfactory efficacy in promoting secretion of TGF-β1 by the NIH/3T3 cells. Both the amino acid mixture and the fish collagen did not show such efficacy.

### C. Wound-Healing Cell Migration Assay:

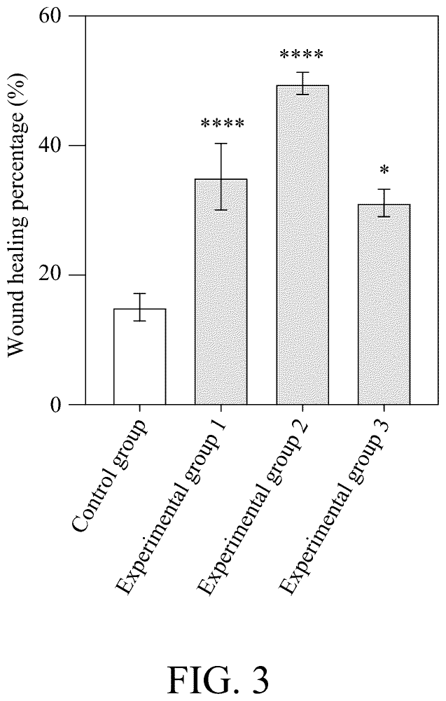

Referring to FIG. 3, the wound healing percentage determined in each of the experimental groups 1 to 3 (treated with the compositions 1 to 3, respectively) was significantly increased compared with that determined in the control group, in which the wound healing percentage determined in the experimental group 2 had the highest degree of increase. These results indicate that the composition according to the disclosure can exhibit satisfactory efficacy in wound healing by promoting fibroblast-mediated wound closure.

Summarizing the above results, it can be seen that use of the amino acid mixture containing glycine and proline or the fish collagen alone not only fails to achieve wound healing effect by promoting attachment of the NIH/3T3 cells and secretion of TGF-β1, but may even cause an adverse effect. In contrast, use of the combination of the amino acid mixture and the fish collagen in the weight ratio ranging from 1:3 to 3:1 (particularly 1:1) can attain extremely excellent effect in promoting attachment of the NIH/3T3 cells and secretion of TGF-β1, and in promoting fibroblast-mediated wound healing. Therefore, the composition according to the disclosure has a high potential to be developed as medicaments for promoting wound healing.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A composition for promoting wound healing, comprising an amino acid mixture and a fish collagen present in a weight ratio ranging from 1:3 to 3:1, the amino acid mixture including glycine and proline.

2. The composition as claimed in claim 1, wherein the amino acid mixture and the fish collagen are present in a weight ratio of 1:1.

3. A method for promoting wound healing, comprising administering to a subject in need thereof a composition as claimed in claim 1.

4. The method as claimed in claim 3, wherein the composition is formulated as a pharmaceutical composition.

5. The method as claimed in claim 4, wherein the pharmaceutical composition further contains a pharmaceutically acceptable carrier.

6. The method as claimed in claim 4, wherein the pharmaceutical composition is in a topical dosage form.
